(19) United States
(12) Patent Application Publication
(10) Pub. No.: US 2009/0149836 A1
(43) Pub. Date: Jun. 11, 2009

(54) Vent-rite

(76) Inventors: Jerald Teachout, Chipley, FL (US); Robert Pearce, Woodstock, IL (US)

Correspondence Address: JERALD TEACHOUT, 4483 HANCOCK CT, CHIPLEY, FL, 32428, US

(21) Appl. No.: 12/008,636

(22) Filed: Jan. 14, 2008

Related U.S. Application Data
(60) Provisional application No. 60/880,493, filed on Jan. 16, 2007.

(51) Int. Cl. A61M 1/00

(52) U.S. Cl. 604/540; 119/200

(57) ABSTRACT

This invention provides an apparatus and method for administration of animal venting therapy in which relieves excess bodily gases (Barotraumas) from the body cavity. The apparatus includes a spring loaded device for making the puncture; this in turn acts as a safety sheath when not in use. A non slip surface is on the front of the device. A main locking device is incorporated for additional safety when the unit is not in use. A soft touch cover for users comfort and with the use of a waist lanyard is additional items added for safety.

### FIELD OF THE INVENTION

This invention relates generally to the field of venting devices, and in particular, to passive venting devices.

### BACKGROUND OF THE INVENTION

Many fish species maintain their depth in the water by adjusting the volume and pressure of gas in their swim bladder in order to maintain neutral buoyancy. When brought to the surface quickly by recreational or commercial fishermen, these fish often suffer from overinflated swim bladders due to the sudden change in water pressure. Some species, such as snapper and grouper have a tendency for the air bladder to force the stomach to protrude from the mouth. If the stomach is punctured, the tear in the lining allows water to penetrate the body cavity of the fish. Death would most likely occur.

These fish floating helplessly on the surface of the water are unable to quickly return to deep water. As a result, they are easy prey for birds and predatory fish. Even if predators don't get them. Releasing the excess gas in the gut cavity of the fish seemed to be the solution to the problem. But teaching anglers how to perform this technique seemed to be a difficult task. In 1994, as a first step in teaching anglers about venting, Florida Sea Grant convened a technical advisory panel to review and develop recommendations aimed at developing educational materials related to gut cavity venting practices.

The panel was comprised of 10 individuals representing fisheries management, marine research, the charter fishing industry, and the Florida Sea Grant Extension Program. The group reviewed current available literature and practices regarding gut cavity venting and discussed the many issues, concerns, and questions related to gut cavity venting.

Commercial, sport, and charter anglers are already venting fish, the problem is, there is a lack of agreement on how and when to vent.

The panel concluded that in many cases, there is an obvious benefit from a properly performed gut cavity venting that allows the fish to overcome surface buoyancy and at the very least swim downward and return to its bottom habitat. What was needed, according to panel members, was a uniform method of venting that could be applied by commercial and recreational anglers.

### SUMMARY OF THE INVENTION

Therefore, what is desired is a passive venting device that is simple and inexpensive to purchase and simple to use. The device will allow for the efficient passive venting of an over inflated swim bladders while preferably eliminating or substantially reducing the lose of life for the reef fishes. There is an obvious benefit from a properly performed gut cavity venting that allows the fish to overcome surface buoyancy and at the very least swim downward and return to its bottom habitat. This device is not just related to the fishing communities, there is also the need for its use in other animal types, like as in the veterinary fields, when there is the problem of animals in the fields and the time for a response from the local vet is overdue long.

### BRIEF DESCRIPTION OF THE DRAWINGS

Reference will now be made, by way of example only, to drawings of the invention, which illustrate the preferred embodiment of the invention, and in which:

FIG. 1 is a perspective view of the passive venting device according to the present invention;

FIG. 2 is a perspective exploded view of the passive venting device according to the present invention;

FIG. 3 is a cross-sectional view of the passive venting device.

FIG. 4 is a top view of the base member of the passive venting device according to the present invention;

### DETAILED DESCRIPTION OF THE PREFERRED EMBODIMENTS

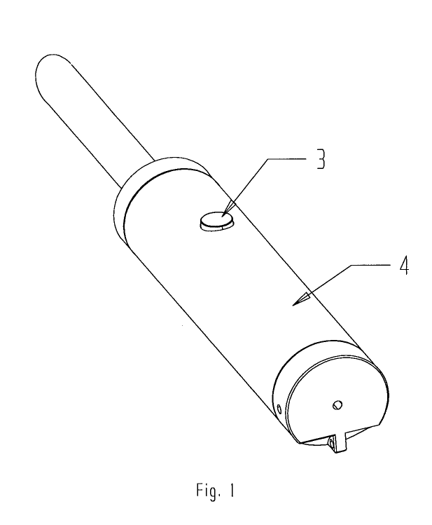

Referring now to the drawings and particularly to FIG. 1 thereof, designated generally by numeral is an embodiment of the animal body cavity venting device according to the present invention. The animal body cavity venting device, comprises of a body of buoyant material, Bright green in color, soft foam handle (4), the external button device (3) that when pushed down its releases the spring load locking device (6).

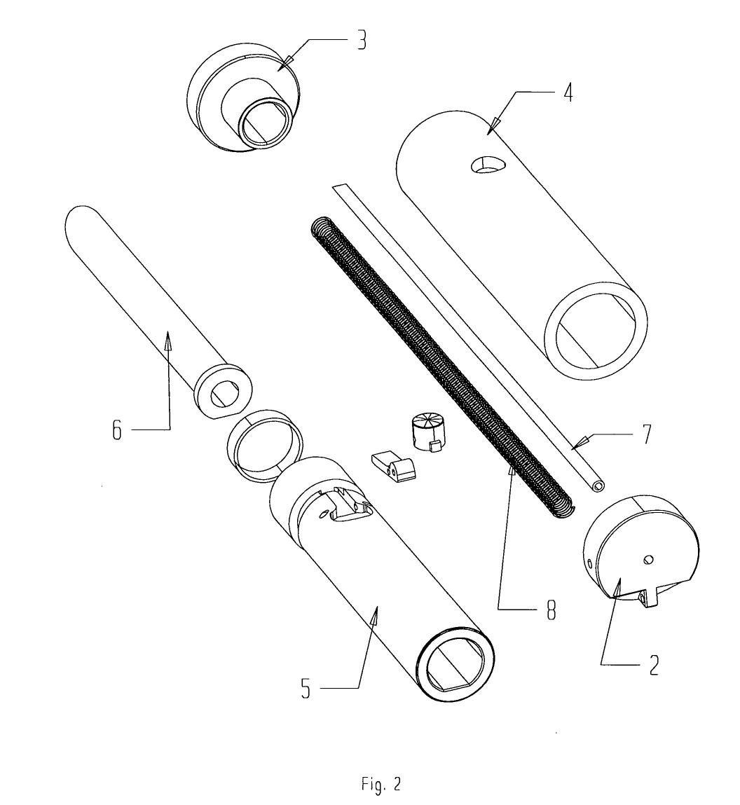

Referring to FIG. 2, the parts of the animal body cavity venting device of this invention, explained within the animal body cavity venting device, this vent needle (7). One end of the vent needle (7) is treaded into an end cap (2) and then tightened to make a sub-assembly. A soft foam handle (4), is slipped over the main body handle (5), this sub-assembly is then fitted with the sub-assembly of the vent needle (7) and end cap (2) and then tightened. This then is then fitted with a spring (8) over the vent needle (7), and then the spring loaded device (6) is slipped under the spring (8) and secured in place with the top end cap (3) and foam handle (4).

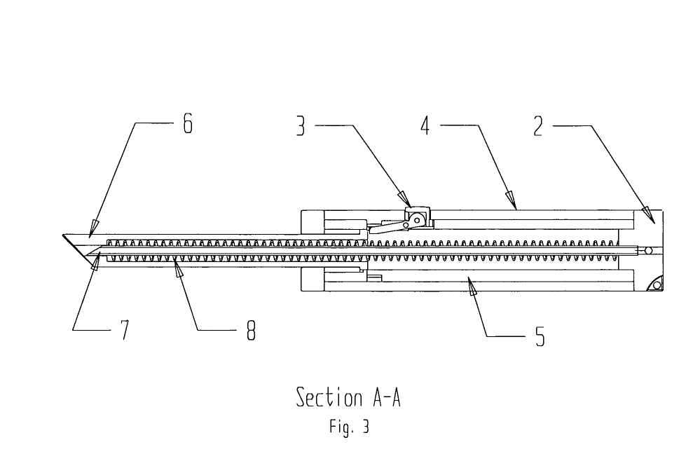

Referring to FIG. 3, exploded view of the animal body cavity venting device (1), the end cap (2), the external locking device (3), the soft foam handle (4), the vent needle (7), the main body handle (5), the spring loaded device (6), and the spring (8).

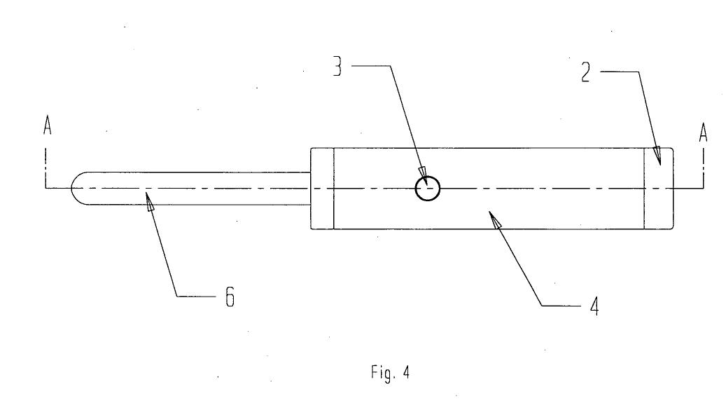

Referring to FIG. 4 a side elevation, the animal body cavity venting device, the end cap (2), the external button device (3), and the spring loaded locking device (6).

The animal body cavity venting device, the end cap (2), the external locking device (3), the soft foam handle (4), and the spring loaded device (6) is made from a plastic material and or metal. The soft foam handle (4) is made from plastic materials.

What is claimed is:

1. A venting device for venting a stomach cavity to the atmosphere, the device comprising, and a method for providing therapy for preventing the death of an animal:

2. The apparatus of claim 1 wherein said operating means is a compression of the spring loaded device to allow the venting device needle to become exposed and the vent device needle is then pushed into said animal to release said gas in the body cavity.

3. The apparatus of claim 1 wherein said vent device needle has a beveled tip to ease the insertion connect to an exit opening to the rear of the device.

4. The apparatus of claim 1 wherein said spring load device has a roughen front surface to aid as a non-slip surface and the spring closing doubles as safety device to cover the vent device needle when not in use.

5. The apparatus of claim 2 wherein said venting device has an internal locking device that when pushed down the spring loaded device of claim 1, the depressing of the external button will release the unit.

6. The apparatus of claim 3 wherein said soft foam grip is mounted to said main body handle of the venting device. Said soft foam grip is of a bright safety color for ease in locating and a non slip soft surface for use with wet and or slippery hands.

7. The apparatus of claim 4 wherein said a lanyard is attached to the rear of the venting device to aid in the handling of said device is some unusual situations.
